(19) United States
(12) Patent Application Publication
(10) Pub. No.: US 2005/0090828 A1
(43) Pub. Date: Apr. 28, 2005

(54) Orthopedic hole filler

(76) Inventor: J. Winslow Alford, Providence, RI (US)

Correspondence Address: Ingrid A. Beattie, Ph.D., J.D.;Mintz, Levin, Cohn, Ferris, Glovsky and Popeo, P.C, One Financial Center, Boston, MA, 02111, US

(21) Appl. No.: 10/911,936

(22) Filed: Aug. 4, 2004

Related U.S. Application Data
(60) Provisional application No. 60/492,461, filed on Aug. 4, 2003.

(51) Int. Cl. A61B 17/56

(52) U.S. Cl. 606/73

(57) ABSTRACT

A method of inhibiting formation of a stress riser in a bone is provided. The method is comprised of providing a bone having a first device and replacing the first device with a second device. Both the first device and the second device are substantially cylindrical, with the diameter of the second device being larger than that of the first device. Formation of a stress riser is inhibited in the presence of the second device.

### RELATED APPLICATIONS

This application claims priority to provisional patent application serial number 60/492,461, filed on Aug. 4, 2003, the entire contents of which are hereby incorporated by reference.

### FIELD OF THE INVENTION

The invention relates to bone implants.

### BACKGROUND OF THE INVENTION

It is often necessary to fix a fractured or broken bone using inserts, for example, screws or other hardware, that are generally constructed of metals. The hardware inserted into the bone can be retained or removed accordingly. There is a significant rate of complications related to retained orthopedic hardware, particularly in high-level athletes in collision sports. Hardware removal can produce screw holes which are stress risers that weaken bone and can require prolonged athlete inactivation to prevent refracture. Refracture following hardware removal can be as high as 26% depending on hardware type and location, and the refracture often occurs through the residual empty screw holes. No intervention to date has effectively minimized the weakening effect of these empty holes.

Patients are often advised to leave asymptomatic hardware in place long after a fracture has healed, because the removal process can be risky. In high performance athletes, however, retained plates and screws have been reported to cause complications. This is especially true in collision sports throughout the world. In a 1 0-year review of National Football League players, there was a 17% refracture rate in football players with well-healed fractures and asymptomatic plates. In this series, the average time to internal fixation was 1.5 days after injury. All of the fractures were internally fixed using standard plating techniques without intra-operative complications. The average time to player reactivation was 18 weeks after surgery. Even with these cautious methods, 17% of the players sustained refracture after reactivation.

A similar survey of rugby players in England revealed a considerable complication rate in players with retained hardware. Athletes in this series who had returned to competitive rugby with retained fracture implants were followed during the period 1990-97. After fracture fixation, the players resumed their preinjury level of participation within one to 12 months. In this series, 13% of these athletes suffered complications in relation to the retained implant.

Routine removal of asymptomatic plates may be a more risky policy than leaving them in. Besides exposing the patient to considerable surgical risk, even successfully removed hardware leaves behind screw holes which act as stress risers, and place the patient at risk of refracture. To prevent refracture following plate removal, the athlete or high demand patient today must endure a prolonged period of mechanical protection prior to his or her full reactivation. The duration of this protected period is of some debate, but recommendations range from several weeks to a full year. As a result, athletes endure a prolonged period of mechanical protection prior to full reactivation. The cost of this inactivation to an athlete's career and his or her team can be significant, particularly in the setting of professional athletes.

For decades, the stress concentration resulting from holes left behind following screw removal has presented a challenge to the orthopedic community. Rates of refracture following hardware removal range from 7% to 26% depending on hardware type and location, with higher rates seen in the forearm following the removal of larger plates, particularly from young and athletic patients.

### SUMMARY OF THE INVENTION

The invention provides an apparatus and procedure for reducing stress risers in bones following orthopedic hardware removal from a bone. The apparatus reduces the risk of fractures associated with empty holes by altering the stress field around the hole. For example, the implant is biocompatible and is optionally bioresorbable, osteoconductive, and/or osteoinductive. The apparatus and procedure of the invention provide a bioresorbable implant that, as the implant breaks down, the implant is replaced by bone growth. An implant, such as a screw, acts as a filler in a bone hole. The implant is substantially cylindrical in shape. In one embodiment, the implant is screw-shaped having threads along substantially the entire length of the implant.

The implant absorbs energy and withstands load similar to bone. The diameter of the implant is slightly greater than the screw hole into which it is fitted and has a tap and screw outer diameter slightly larger than the screw hole to allow for complete filling of the screw hole. The bioresorbable implants are an array of lengths to accommodate varying bone thickness. The implant device is unicortical or bicortical in that it links two cortices of a bone.

Implementations of the invention may include one or more of the following features. The bioresorbable implant is created from a combination of PLA/PGA (Polylactic Acid/Polyglycolic Acid). The implant is composed of, for example, 82% PLA and 18% PGA to modulate the degradation rate and promote resorption as bone replaces the implant material. Additionally, the bioresorbable implant includes TCP (Tricalciumphosphate). The implants absorb water, which has the effect of causing the implant to swell, thereby substantially completely filling the screw hole.

In another aspect, the invention provides a method of inhibiting stress riser in a bone. The method includes providing a bone having a first device and replacing the first device with a second device, wherein both the first device and the second device are substantially cylindrical. The diameter of the second device is larger than the diameter of the first device, and formation of a stress riser is inhibited in the presence of the second device. Alternatively, the diameter of the second device is substantially the same or less than that of the first device, and the second device expands to a larger diameter upon contact with a stimulus such as moisture or heat (e.g., conditions encountered upon insertion into a bodily tissue or cavity). In another example, the second device bites into bone as it is screwed into the residual hole left following removal of the first device. In yet another example, the hole filler is a two piece device in which a first part (the diameter of which is substantially the same or smaller than the first device) is inserted into the residual hole left following removal of the first device, and a second piece (e.g., a wedge-shaped piece) is inserted into the first piece causing the first piece to expand. The first device is a device with which the patient presents following a surgical repair of a bone fracture. The first device was inserted into the bone at the time of surgical repair and which is to be removed and replaced by the second device.

Implementations of the invention may include one or more of the following features. The first device is non-porous metallic and the second device is preferably non-metallic or porous metallic. For example, the second device is biodegradable. The second device comprises a solid composition, wherein the solid composition is substantially elastic. The second device can comprise a non-rigid solid composition. The second device further comprises a polymer. The second device comprises a polylactic acid. The second device comprises a polygalactic acid. Alternatively, the second device comprises a polylactic acid and a polygalactic acid. In some embodiments, the second device is ceramic or a ceramic/metal biocomposite. For example, the second device further includes tricalcium phosphate as at least a portion of its composition. In other embodiments, the second device contains a nickel-titanium alloy (e.g., Nitinol) or a porous trabelcular metal such as tantalum (e.g., Hedrocel®). The second device is formed in the shape of a screw.

The device of the invention provides one or more of the following advantages compared to earlier methods. The biodegradable implant is effective as an implant in a weight bearing bone such as a femur, a tibia bone, or a non-weight bearing bone such as a forearm bone, e.g., a radius or ulna bone. The implant is applicable in any bone that has undergone hardware removal to leave an empty hole. Use of a growth factor in addition to the polymer composition of the implant stimulates and improves bone growth that eventually substantially completely replaces the implant. Filling bone holes with bioresorbable implants leads to at least 10%, 25%, 50%, 75%, or 90% increase in the amount of energy absorbed prior to failure. For example, a PLA hole filler led to 73% increase in the amount of energy absorbed prior to failure. Other higher percentages of increase in energy absorption are possible. Bones filled with the filler implant withstand a higher maximum torque than bone without the filler. The mean increase in the maximum torque that the bone having the filler is able to withstand increases by 10%, 20%, 30%, 50% or more. The bioresorbable implants additionally comprise characteristics that measurably improve upon the maximum torque to failure in a bone, energy to failure, fracture characteristics of bones. Growth factors and bone morphogenetic factors are optionally incorporated into or onto the device to enhance progressive bone ingrowth.

### BRIEF DESCRIPTION OF THE FIGURES

FIG. 1 is a perspective drawing of a bioresorbable bone screw according to one embodiment of the invention;

FIG. 2 is an exemplary illustration of an anterior cortex of a bone having a screw hole and showing fracturing;

FIG. 3 is an exemplary illustration of a posterior cortex of a bone having a screw hole and showing fracturing;

FIG. 4 is an illustration of an anterior cortex of a bone having a bioresorbable implant according to one embodiment of the invention;

FIG. 5 is an illustration of a posterior cortex of a bone having a bioresorbable implant according to one embodiment of the invention;

FIG. 6 is a perspective drawing of an embodiment of the invention;

FIG. 7 is a bar graph of the percentage change of load, energy and stiffness using a metal implant; and

FIG. 8 is a bar graph of the percentage change of load, energy and stiffness using a bioresorbable implant in one embodiment of the invention.

FIG. 9 is a photograph of a device with differential screw pitch.

### DETAILED DESCRIPTION OF PREFERRED EMBODIMENTS

Empty holes left after removal of a first device, e.g., a metal pin or rod, are filled with a second device, e.g., a hole filler such as a bioresorbable implant or porous metallic implant which fill in empty holes left following metal hardware removal from the bones.

Embodiments of the invention are directed to an osteoconducfive, osteoinductive, bioresorbable, or biodegradable, implantable bone filler or screw used subsequent to removal of metal hardware that is present in a hole in the bone due to an orthopedic procedure. In one example, the implant has bioactive effects on ossification, such as recruitment of mesenchymal cells by growth factors in the implant (osteoinductive properties). Alternatively or in addition, the implant provides a three-dimensional framework for the ingrowth of capillaries and osteoprogenitor cells (osteoconductive properties).

The apparatus of the invention alters the stress field around the hole by expanding, mechanically or chemically, and the expansion of the apparatus reduces the risk of refracture associated with holes. The invention can be used for a number of orthopedic procedures and for purposes other than bone filling after hardware removal. Still other embodiments are within the scope of the invention.

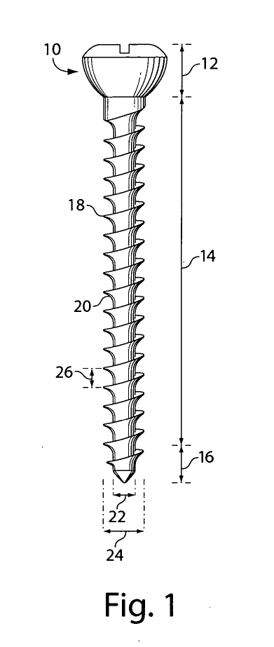

Referring to FIG. 1, a bioresorbable screw 10 for use in a bone having a screw hole is shown. The bioresorbable screw 10, also referred to as a filler or an implant throughout, includes a head 12, a shank 14, a tip 16, and threads 18. The threads 18 have a thread angle 20 and a pitch 26. The shank 14 has a core diameter 22 and an outer diameter 24. The head 12 has a diameter 13 and can be substantially flat in shape. The head 12 can include a torsion-control fail mechanism. The core diameter 22 and the outer diameter 24 of the shank 14 are dictated by the width and depth of the hole into which the bioresorbable screw 10 is inserted. The thread pitch 26 can also vary. The bioresorbable screw can have a core diameter 22 and outer diameter 24 slightly larger than the hole into which the screw is inserted to ensure that the hole is substantially completely filled by the screw. Preferably, for example, the screw can have a core diameter 22 of 2.7 mm, an outer diameter 24 of 3.7 mm, a thread pitch of 1.25 mm, and a head diameter 13 of 6 mm. Because bones vary in thickness, the screw is additionally available in an array of lengths to accommodate the varying thickness of bones.

The bioresorbable screw 10 is composed of a mixture of Polylactic Acid (PLA) and Polyglycolic Acid (PGA). The composition may further include Tricalcium phosphate (TCP). The use of other polymers is also possible. The combination of PLA/PGA and TCP modulates the rate of degradation and permits bone ingrowth, or osteoconduction. A combination of 82% PLA and 18% PGA, for example, can comprise a bioresorbable screw that effectively modulates a rate of degradation of the screw 10 in conjunction with a rate of osteoconduction in the bone. Other percentage combinations of PLA and PGA are also suitable. The bioresorbable screw 10 can additionally include biologically active substances, such as growth factors. The growth factors include, but are not limited to, Bone Morphogenic Protein-6 (BMP-6 and BMP-7), which provide bone formation stimulation, or osteoinduction.

A number of additional polymers can be used in the composition of the implant 10. More than 40 different biodegradable polymers are known, only some of which are used in orthopedic surgery. In the field of operative sports medicine, the poly-α-hydroxy acids such as PLA and PGA, including their copolymers and stereopolymers are most frequently used. The degradation of chains of synthetic biodegradable polymers consisting of poly-hydroxy acids results from an unspecific hydrolysis as the implant absorbs water. Lactic acid polymers, for example, are reduced to monomers which are in turn dissimilated to carbon dioxide and water via the Krebs Cycle. The ability of regional tissues to process the lactic acid accumulation is determined by the implant size, rate of implant degradation, and the polymer type. Some polymers are associated with rapid degradation and the creation of sterile abscesses and osteolysis, caused by a regional overload of lactic acid during degradation, releasing prostaglandins and other inflammatory mediators.

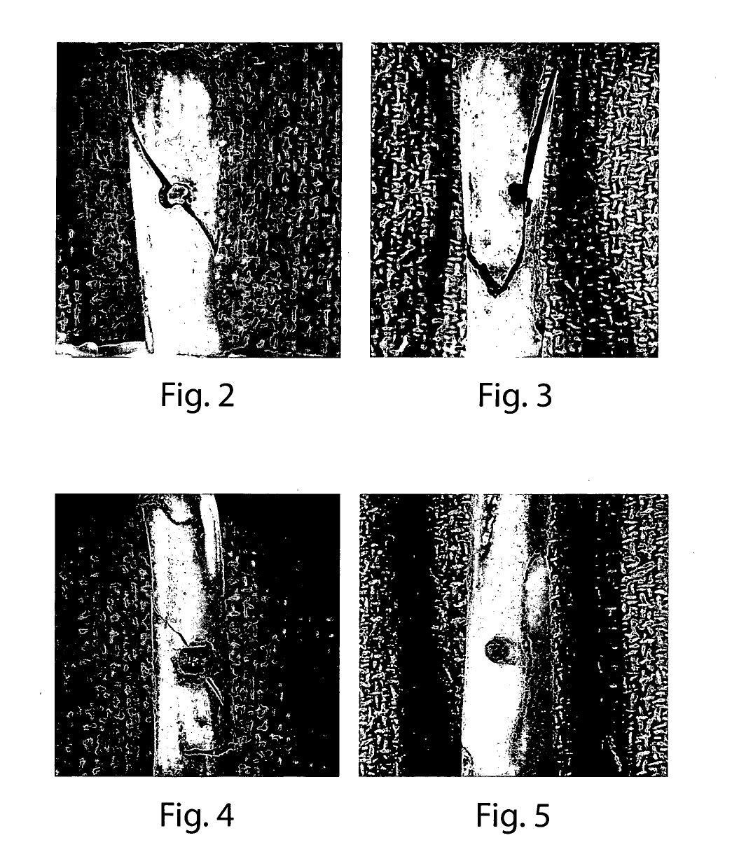

The bioresorbable screw 10 of FIG. 1 is preferably composed of 82% PLA and 18% PGA, a ratio which modulates the degradation rate and promotes more predictable resorption as bone slowly replaces the screw material. Other ratios are possible and envisioned. The PLA/PGA screw 10 swells slightly as it absorbs water, which enhances mechanical strength. The swelling, or expansion of the screw 10 alters the stress field around a bone hole to strengthen the bone in that area. The mechanics of the screw 10 are particularly advantageous given known effects of torsion on a long bone, described below in conjunction with FIGS. 2 and 3.

Referring to FIG. 2 and FIG. 3, anterior and posterior views of a bone having an aperture, or bone hole are shown. FIG. 2 is an anterior perspective of a bone 30. The bone 30 is shown having a screw hole 32 and a fracture 34. FIG. 3 is a posterior perspective of the bone 30 of FIG. 2. The screw hole 32 extends through the width of the bone 30, thereby open to both the anterior and the posterior cortices of the bone 30. The bone fracture 34 extends at a 45-degree angle from the anterior cortex surface of the screw hole 32 to the posterior cortex surface of the screw hole 32.

Further referring to FIGS. 2 and 3, torsion of a bone causes forces to be distributed into regions of pure tension and pure compression. The vectors of shear and compression are oriented perpendicular to one another in the plane of the cortex surface. As cortical bone is weaker in tension than compression, the first failure occurs in tension. A first failure of the bone is represented in FIG. 2 by bone fracture 34, which extends substantially 45 degrees from the long axis of the bone 30. Once the fracture 34 initiates, it propagates along a 45-degree angle spiral path to the opposite, or posterior cortex, for example, which then fails in compression.

In FIGS. 4 and 5, the screw hole in a bone is filled with an implant. The implant 50 rests in a screw hole 52 of a bone 54. The bone can be, for example, a weight bearing bone, such as a femur, or a non-weight bearing bone, such as a forearm bone. FIG. 4 depicts an anterior cortical view of the implant 50 in the screw hole 52, while FIG. 5 depicts a posterior cortical view of the same implant 50 in the screw hole 52, which extends through the width of the bone. Stress risers are present on the anterior and posterior cortices of the bone. Filling the screw hole 52 results in a bone fracture 56 that as it propagates, misses the opposite, or posterior cortex. The bolstering effect of the implant 50 redirects the fracture propagation away from the stress riser, into intact bone. The bone 54, therefore, absorbs more energy prior to failure. Additionally, the presence of the screw 50 biomechanically links the anterior cortex and the posterior cortex, essentially requiring that the whole bone fail as a single system, thereby absorbing more energy prior to failure. A still further mechanism of the protective effect of filling the holes 52 with the screw 50 is that the act of screw replacement pre-stresses the holes and limits the local stress rising effect.

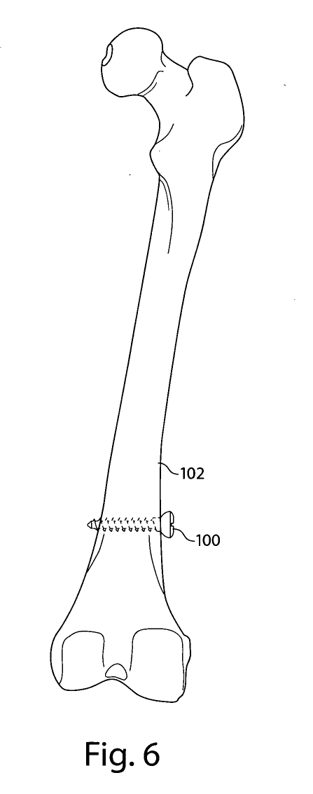

Referring to FIG. 6, a perspective view of an alternative embodiment of the present invention is shown. A bioresorbable implant 100 is inserted into a bone 102. The implant 100 is inserted across both cortices of the bone. The implant 100 expands beyond the diameter of the hole on the outside of the bone, filling the space within the bone, and linking the cortices. FIG. 6 represents an implant in a shape other than a screw. Other embodiments are also envisioned.

Differential Screw Pitch

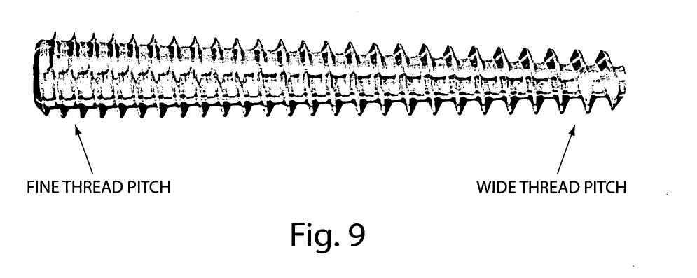

Throughout its length, the device is manufactured to have a screw pitch which is of differential pitch (See FIG.9). A wide thread pitch at the leading tip of the screw advances the device more rapidly compared to the trailing end of the screw with finer threads. This configuration causes compression as the screw crosses the two cortices of the bone, thereby linking the anterior and posterior cortices. Screw pitch refers to the angle of the thread relative to the length of the screw. The leading tip (wider end) of the screw differs between 1-10% in screw pitch compared to the narrow (trailing) end of the screw. Preferably, the difference is between 2-7%.

Torque Control Screw Head

For screw-in devices, the screw is adequately but not excessively advanced into the bone hole. To assure that the proper amount of torque is applied to the screw, the neck of the screw (the region under the screw head) is designed to fail when a predetermined amount of torque is applied, leaving the screw implanted in bone as a low profile headless screw. The amount of force or torque required depends on the size of the bone to be repaired and the screw to be used. Typically, three different screw types/sizes are employed: a small screw (about 2.8 mm average diameter) for small bones such as hand (metacarpal) bones; a medium screw (about 3.7-3.8 mm average diameter) for medium bones such as forearm or lower leg bones; and a large screw (about 4.8 mm average diameter) for large bones such as a femur. For a small bone (and screw), the head fails in the range of 0.25-2 Newton-meters (Nm) of torque, preferably in the range of 0.5-1.5 Nm of torque, and most preferably at about 1 Nm of torque. For a medium-sized bone (and screw), the head fails in the range of 0.5-15 Nm, preferably in the range of 3-7 Nm, and most preferably at about 4 Nm. For a large bone (and screw), the head fails in the range of 4-12 Nm, preferably in the range of 5-10 Nm, and most preferably at about 7 Nm.

Inhibition of Stress Risers in a Femur

The mechanical effect of defect (screw hole) filling was tested using paired rabbit femurs, one of which was filled with either a metal or a bioresorbable bone screw, and the other left empty. Thirty paired rabbit femora were carefully cleaned of soft tissue, and the bone ends were potted with PMMA in short lengths (2.54 cm) of square aluminum tube stock. After potting, a single 2 mm (20% of cortical diameter) bicortical hole was drilled through the femoral mid-shaft in the anterior-posterior direction of 28 of the paired femurs.

Two of the 30 paired femurs were potted in an identical manner, left undrilled and tested to establish a baseline for comparison. The remaining 28 paired femurs were randomly divided into two experimental groups, and the hole in one randomly-selected bone in each pair was filled with either a standard 2 mm AO stainless steel screw (Synthes, Paoli, Pa.) or a 2 mm bioresorbable screw (82% polylactic acid (PLA), and 18% polyglycolic acid (PGA), manufactured by Biomet, Inc., Warsaw, Ind.). Prior to screw insertion, the holes were tapped using a manufacturer-supplied tap, which was specific to the screw type. The screws were used as fillers, and inserted through both cortices. The empty holes in the contralateral control bones were also tapped to match the holes in the filled bones. These specimens were tested as pairs to accommodate for slight variations between the rabbits anatomy, and the instruments which were specific to screw type.

All of the bones were tested to failure in external rotation and the data was reduced to determine the maximum torque to failure and the total amount of energy absorbed by the bone prior to failure. In addition, the type of fracture was noted (spiral, transverse or comminuted), as well as the angle of the fracture relative to the long axis of the bone (measured with a miniature goniometer) and whether the fracture passed through the anterior and/or posterior holes.

Placing a metal screw in the diaphyseal hole produced a mean 17% increase in maximum torque (from 1.68 Nm to 1.97 Nm), and a 58% increase in the amount of energy to failure (5.66 Nmm to 8.97 Nmm). A bioresorbable screw filler produced a mean increase of 30% in the maximum torque (from 1.41 Nm to 1.84 Nm) and a 73% increase in the amount energy to failure (3.38 Nmm to 5.86 Nmm). These differences were all statistically different by Student's T-test, with p<0.05.

Due to accidental fracture of 2 bones prior to testing, 26 bones remained in the study. Of the 26 bones remaining, 13 were filled with PLA or metal screws and 13 were filled with empty mid-diaphyseal holes. A survey of fracture characteristics demonstrated that all fractures occurred in a spiral pattern at a 45°0 (±2°) angle to the long axis of the bone. All fractures included at least one of the two cortical defects created by the single mid-diaphyseal screw hole. If a screw hole was filled, the fracture was more likely to miss one of the two possible cortical defects. In total, the fractures of only 4 of 13 filled bones passed through both cortical defects, whereas the fractures in 11 of 13 bones with empty holes passed through both cortical defects. This difference was a statistically significant value of (P<0.01) by Chi Square.

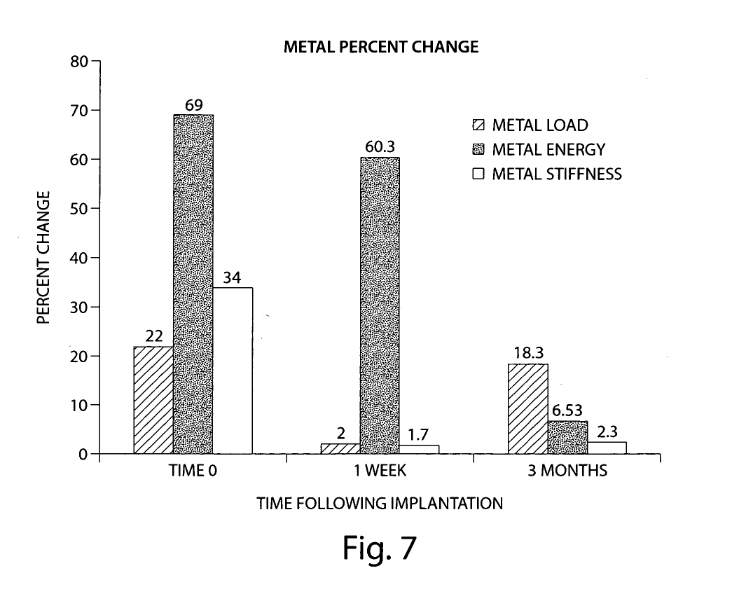

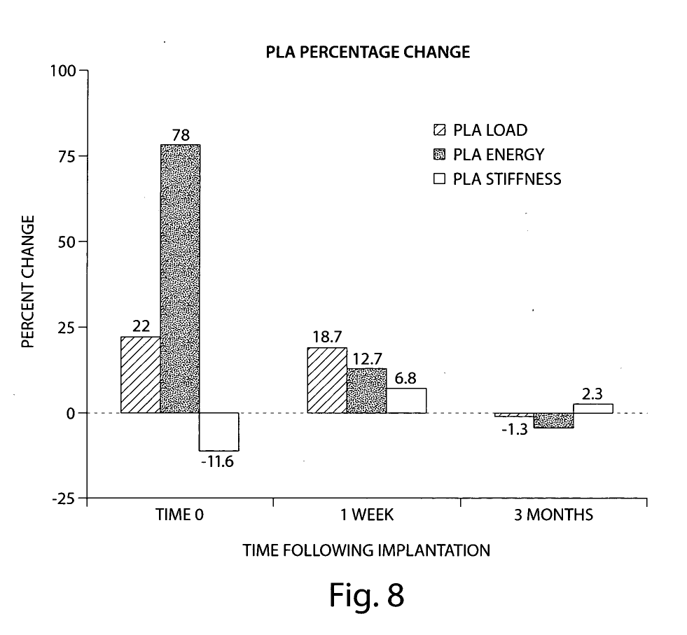

In the bar graphs of FIG. 7 and FIG. 8, the change in load, energy, and stiffness of implants are depicted. Referring to FIG. 7, the percentage of change in a metal implant versus an empty bone having no implant is displayed. The percentage of change in the metal load, metal energy, and metal stiffness are charted at particular time periods following implantation, specifically at the time of implantation, one week following implantation, and three months following implantation. Likewise, referring to FIG. 8, the change in load, energy, and stiffness of a PLA, or biodegradable implant is graphed. Percent change refers to the change in physical properties of a bone with an empty hole (e.g., a hole left after removal of a metallic device) compared to bone containing a hole filler implant (i.e., the void of the empty hole replaced with an implant described herein). The implant was found to absorb substantially more energy.

Filling the screw hole with a bioresorbable screw reduces the stress-riser effect of a screw hole. The data described herein demonstrates an immediate protective effect of filling residual screw holes following hardware removal by allowing the bones with filled screw holes to absorb more energy prior to failure and to withstand a higher maximum torque than their matched pairs with empty holes.

Thus, filling a mid-diaphyseal hole with a bioresorbable screw substantially immediately reduces the stress riser caused by the empty hole. Because refractures usually occur shortly after plate removal, the protective effect of the bioresorbable screw in increasing the maximum load to failure and in its capacity to absorb energy reduces the incidence of refracture in patients following hardware removal.

The data presented involves cadaver bones subjected to pure torsional forces; actual injuries occur in larger bones, at higher energy levels in a combination of torsion and compression. Despite this limitation, the carefully controlled nature of the testing allowed isolation of the effect of the filled screw holes, revealing an intriguing protective effect of the filled vs. empty holes, even at low magnitude forces. These mechanical features apply to bones of any size. Because a large percentage of the refractures following hardware removal occur acutely, the purely mechanical protective effect observed is of particular interest because immediately after hardware removal, an increase in energy absorption prior to failure is achieved through mechanical, rather than biological means.

An improvement achieved by using biodegradable fillers in a bone hole is that it provides an immediate strengthening effect at the time of implantation. The data herein indicate that immediately following hardware removal, a bone gains immediate strength if the removed screws are replaced by a hole filler such as PLA/PGA fillers, and this increased strength raises the threshold for refracture.

### Other Embodiments

Embodiments of the invention describe biodegradable bone fillers for use in reducing the stress-riser effect of a screw hole. Other configurations are possible, such as configurations of a biodegradable bone filler in a shape other than a screw-shape, such as a smooth cylinder, a ribbed cylinder, or other shapes that can be envisioned. Further embodiments are possible, such as a bone filler fabricated of porous metal, which is any of a number of metallic materials having microscopic pores. The pores allow for microscopic ingrowth of bone and incorporation of the implant into native bone, rather than by replacing the bone, known as osteoconduction. Both the porous metal filler and the biodegradable filler can comprise a number of shapes other than screw-shaped fillers.

Additionally, embodiments of the invention describe methods of using a biodegradable bone filler for use in a bone hole from which a first metal bone filler was used for orthopedic procedures, but has been removed. Other embodiments of the invention can be used for purposes other than following orthopedic hardware removal, such as procedures wherein it is desirable to avoid stress risers, such as in tumor resection, cyst removal, bullet hole treatment, or other treatment of other pathological lesions.

Embodiments of the invention can also include a biodegradable or a porous metallic implant comprised of a second implant portion in addition to the first implant portion. The second implant portion can be used to cause the first implant portion to expand slightly, acting similar to a wedge, such that the first implant portion continues to effectively prevent stress risers in the bone.

The invention can be embodied in other specific forms without departing from the spirit or essential characteristics thereof. The foregoing embodiments are, therefore, to be considered in all respects illustrative rather than limiting on the invention described herein.

Having thus described at least one illustrative embodiment of the invention, various alterations, modifications and improvements will readily occur to those skilled in the art. Such alterations, modifications and improvements are intended to be within the scope and spirit of the invention. Accordingly, the foregoing description is by way of example only and is not intended as limiting. The invention's limit is defined only in the following claims and the equivalents thereto.

1. A method of inhibiting formation of a stress riser in a bone, comprising providing a bone, said bone comprising a first device and replacing said first device with a second device, wherein said first device and said second device are substantially cylindrical, the diameter of said second device being larger than that of said first device and wherein formation of a stress riser is inhibited in the presence of said second device.

2. The method of claim 1 wherein said first device is metallic and said second device is non-metallic.

3. The method of claim 1, wherein the second device is biodegradable.

4. The method of claim 1, wherein the second device is non-biodegradable.

5. The method of claim 1, wherein the second device is replaced by bone or incorporated by native bone.

6. The method of claim 1, wherein the second device comprises a non-rigid solid composition.

7. The method of claim 1, wherein the second device comprises a polymer.

8. The method of claim 1, wherein the second device comprises a polylactic acid.

9. The method of claim 1, wherein the second device comprises a polyglycolic acid.

10. The method of claim 1, wherein the second device comprises a polylactic acid and a polyglycolic acid.

11. The method of claim 10, wherein the second device further comprises tricalcium phosphate.

12. The method of claim 1 wherein the second device comprises a porous metal.

13. The method of claim 1, wherein said bone is a weight bearing bone.

14. The method of claim 1, wherein said bone is selected from the group consisting of a femur, a tibia and a forearm.

15. The method of claim 1, wherein said second device is in the shape of a screw.

16. The method of claim 15, wherein said second device has a length, and wherein threads extend along the length of the second device.

17. A method of inhibiting refracture of a previously fractured bone, comprising filling an aperture in said bone, said aperture having been occupied by a first device and wherein filling said aperture comprises filling with a second device, and wherein refracture of said bone is inhibited in the presence of said second device.

18. The method of claim 17, wherein said first device and said second device are substantially cylindrical and wherein the diameter of said second device is greater or can expand to become greater than that of said first device.

19. The method of claim 17, wherein the second device is biodegradable.

20. The method of claim 17, wherein the second device is non-biodegradable.

21. The method of claim 17, wherein the second device is replaced by bone.

22. The method of claim 17, wherein the second device comprises a non-rigid solid composition.

23. The method of claim 17, wherein the second device comprises a polymer.

24. The method of claim 17, wherein the second device comprises a polylactic acid.

25. The method of claim 17, wherein the second device comprises a polyglycolic acid.

26. The method of claim 17, wherein the second device comprises a polylactic acid and a polyglycolic acid.

27. The method of claim 17, wherein said bone is a weight bearing bone.

28. The method of claim 27, wherein said weight bearing bone is selected from the group consisting of a femur and a tibia.

29. The method of claim 17, wherein said second device is in the shape of a screw.

30. The method of claim 29, wherein said second device has a length, and wherein threads extend along the length of the second device.

31. A bicortical device for inhibiting a stress riser in a bone, the bicortical device linking two cortices of a bone and comprising a non-metallic expandable composition having varying lengths that correspond to a relative thickness of said bone, and the bicortical device further comprising a cylindrical shaft portion for implantation into said bone.

32. The bicortical device of claim 31, wherein said cylindrical shaft portion comprises threads along the length of said shaft portion.

33. The bicortical device of claim 31, wherein said non-metallic expandable composition is a biodegradable material.

34. The bicortical device of claim 31, wherein said non-metallic expandable composition is a non-biodegradable material.

35. The bicortical device of claim 34, wherein said non-metallic expandable composition comprises a solid having elastomeric properties.

36. The bicortical device of claim 31, wherein at least a portion of a composition of the device is comprised of a polymer.

37. The bicortical device of claim 31, wherein at least a portion of a composition of the device is comprised of a polylactic acid.

38. The bicortical device of claim 31, wherein at least a portion of a composition of the device is comprised of a polyglycolic acid.

39. The bicortical device of claim 31, wherein at least a portion of a composition of the device further comprises tricalcium phosphate.

40. The bicortical device of claim 31, wherein said non-metallic expandable composition includes a polylactic acid and a polyglycolic acid.

41. The bicortical device of claim 31, wherein said bone is a weight bearing bone.

42. The bicortical device of claim 31, wherein said weight bearing bone is selected from the group consisting of a femur, a tibia, and a forearm bone.
